(19) United States
(12) Patent Application Publication
(10) Pub. No.: US 2019/0236736 A1
(43) Pub. Date: Aug. 1, 2019

(54) ADVANCED CARE PLANNING PROCESS

(71) Applicant: Hana Malik, Lombard, IL (US)

(72) Inventor: Hana Malik, Lombard, IL (US)

(21) Appl. No.: 15/882,000

(22) Filed: Jan. 29, 2018

(51) Int. Cl. G06Q 50/18; G16H 10/60; G06Q 10/10

(52) U.S. Cl. CPC G06Q 50/186; G06Q 10/10; G16H 10/60

(57) ABSTRACT

A computerized process for advanced care planning of a customer user includes, within a computerized processor, operating programming configured to monitoring designation by the customer user of end of life wishes of the customer user. Monitoring designation of the end of life wishes includes at least one of monitoring entry of a durable power of attorney agent, monitoring entry of choices related to a living will for the customer user, and monitoring entry of choices related to last wishes of the customer user. The process further includes monitoring entry of at least one designee to act on behalf of the customer user, securely holding the end of life wishes until a designated event occurs, and releasing the end of life wishes to the designee upon occurrence of the designated event.

### TECHNICAL FIELD

This disclosure is related to a computerized process for medical care planning.

### BACKGROUND

The statements in this section merely provide background information related to the present disclosure. Accordingly, such statements are not intended to constitute an admission of prior art.

People make plans for illness, incapacitation or infirmity, and for estate planning. Living wills are known to provide direction for medical care if the person is not capable of making choices about care. Last wishes document end of life choices for a person. Wills provide direction for where inheritance is to be directed when the author of the will passes away. Wills and living wills are frequently generated in paper format. Such paper documents must be found in order to be executed.

Online programs are known for providing guidance in financial or medical issues. TurboTax® and QuickBooks® provide step by step instructions to taxpayers and business owners, respectively, to get professional advice and prepare paperwork that previously might have been done on paper. Medical insurance websites help customers to make selections between various insurance plans, evaluate coverage and deductibles, plan in network vs. out of network care, and engage contractual coverage.

### SUMMARY

A computerized process for advanced care planning of a customer user includes, within a computerized processor, operating programming configured to monitoring designation by the customer user of end of life wishes of the customer user. Monitoring designation of the end of life wishes includes at least one of monitoring entry of a durable power of attorney agent, monitoring entry of choices related to a living will for the customer user, and monitoring entry of choices related to last wishes of the customer user. The process further includes monitoring entry of at least one designee to act on behalf of the customer user, securely holding the end of life wishes until a designated event occurs, and releasing the end of life wishes to the designee upon occurrence of the designated event.

### BRIEF DESCRIPTION OF THE DRAWINGS

One or more embodiments will now be described, by way of example, with reference to the accompanying drawings, in which:

FIG. 1 illustrates an exemplary server device configured to operate a system administration portion of an advanced care planning process, in accordance with the present disclosure;

FIG. 2 illustrates an exemplary portable computerized device configured to operate a user portal portion of an advanced care planning process, in accordance with the present disclosure; and

FIGS. 3A-C illustrate a flowchart illustrating an exemplary computerized process configured to operate an advanced care planning process, in accordance with the present disclosure.

### DETAILED DESCRIPTION

A computerized process for advanced care planning. Such a process can provide, through a desktop computer, smart phone or other computerized device, access to an online server providing the computerized process as a service.

Exemplary, non-limiting details of one embodiment of the disclosed process are provided. The discloses process and the system operating that process include a web-based, on-line program allowing user to create an advance directive by following self-directed prompts and options. The user creates a profile with information including personal contact info and medical provider information. In one embodiment, the user assigns durable power of attorney. In one embodiment, the user selects preferences for end of life medical care, including end of life personal care and end of life care arrangements. In one embodiment, the user has option to create or import a medical chart. In one embodiment, the user can provide information to enable automatic billing to insurance and/or a medical service provider.

In one embodiment, the user can enable and configure automatic email to family and friends, either based upon a scheduled time or in response to some event taking place. The user can record private video message, voice message, write and electronic letter to be stored (e-vault) for retrieval and delivery at a later time.

A completed application can be printed, emailed and downloaded in electronic form. A completed application will be downloaded to home screen of smart phone and can be accessed without having to enter password on phone. Such a feature can be described as a lock screen widget feature.

The disclosed system can enable a company to maintain a national registry or state-specific registry (on-line database) of living wills and vital information data files.

The disclosed system can provide a secure-online database providing our members with immediate access to a copy of their living will, advance directive and durable power of attorney for health care. The database and associated server can provide for storing and preparation of living wills and designation of healthcare surrogates and other legal documents. Access to the database can be provide based upon an identifying PIN, thumb print recognition, or other similar means.

The disclosed process can provide a sticker for a user's driver's license, ID card and or insurance card and wallet card alerting emergency personnel of advanced directives.

The disclosed process and system can provide educational or advisory materials, aiding a user to make educated decisions about the provided services. In one example, the process can provide videos explaining end of life options and options for estate planning. In another example, an attorney or professional can be made available through video conference or through chat messaging to answer questions.

The disclosed system enables one to go through secure identity confirmation, and the user can then edit his or her plan.

The disclosed system can permit a user to configure regular reminders at a selected frequency via text and email for user to update advance directives.

The system can utilize e-signature capability to receive signatures. Automatic requests for electronic signature can be utilized based upon technical and legal requirements related to estate planning and advanced care directives.

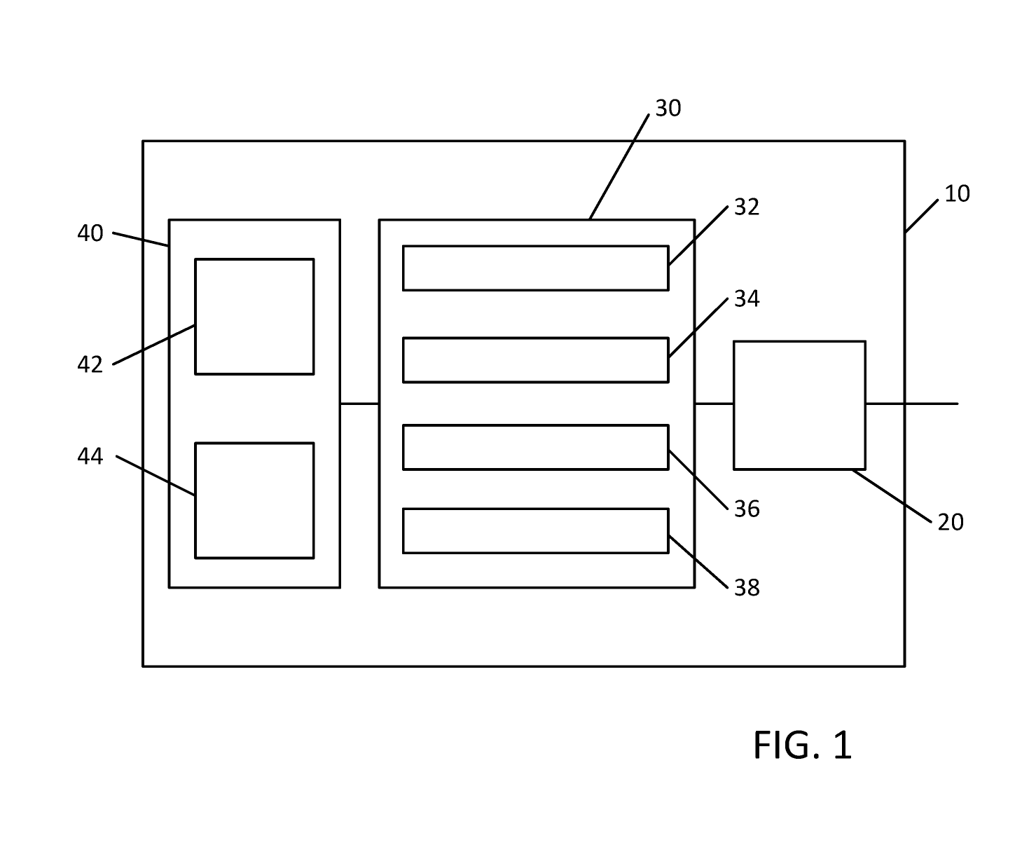

Referring now to the drawings, wherein the showings are for the purpose of illustrating certain exemplary embodiments only and not for the purpose of limiting the same, FIG. 1 illustrates an exemplary server device configured to operate a system administration portion of an advanced care planning process. Server 10 is illustrated including a communications device 20, a processor device 30, and a storage device 40. Server device 10 is exemplary and can include any alternative computerized device capable of operating an online service or website. Server device 10 includes at least one computerized processor configured to operate processes as computerized code, random access memory, and memory configured to store data. Server device 10 can include a single physical device or can be embodied as a plurality of devices providing the combined functionality.

Communications device 20 includes a device configured to send and receive information over a communications network. Communications device 20 is configured to provide communicative connections between server 10 and a plurality of users simultaneously making use of the processes provided by server 10.

Processor device 30 includes a computerized processor and operates computerized processes embodied as one or more computerized modules 32, 34, 36 and 38 including code configured to perform certain functions enabling the disclosed process.

User identification and interface module 32 is configured to interact with users communicating with server 10, receive identification data, process password entry and recognition, provide registration functions, generate email messages, and other similar functions. Living will/medical wishes process module 34 is configured to provide functionality configured to enable users to set up and maintain choices in the database and further provide notice of the choices to appropriate medical facilities and providers. Last wishes/estate disposition module 36 is configured to provide functionality configured to enable users to set up and maintain published final wishes and/or estate planning choices in the database and further provide notice of the wishes or choices to the necessary people or organizations. Data vault module 38 provides programming configured to securely hold and maintain secret data related to the choices and wishes of each user, providing access to the data to only the appropriate people at selected times.

Storage device 40 can include an exemplary hard drive device and includes an exemplary user/entity database 42 and a choices and wishes database 44. User/entity database 42 includes data related to customer users to the disclosed process, medical facility login and interaction data for facilities that provide care for the users, legal professional login and interaction data for professionals and court related entities to interact with the system, registered witness login and interaction data, and other relevant data necessary to provide authorized access and data flow related to the disclosed process. Choices and wishes database 44 can include data, completed questionnaires, will documents, and other related documentation related to the selections made by users in the disclosed process. Choices and wishes database 44 can include additional safeguards as compared to user/entity database, for example, requiring administrative approval, registered email confirmation, two-factor authentication, or other rigorous processes known in the art to prevent accidental disclosure of the choices and selections recorded by the users.

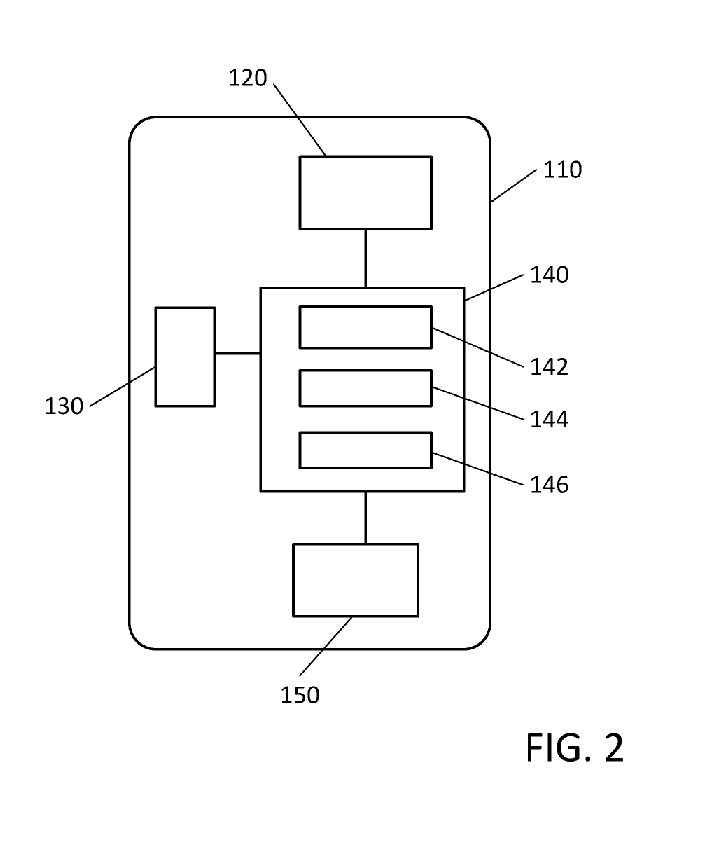

FIG. 2 illustrates an exemplary portable computerized device configured to operate a user portal portion of an advanced care planning process. Portable computerized device 110 is provide as an exemplary smart phone device and can include any alternative computerized device capable of operating a consumer based computer application or program. Portable computerized device 110 includes a communications device 120, a user interface device 130, a processor device 140, and a storage device 150. Portable computerized device 110 includes at least one computerized processor configured to operate processes as computerized code, random access memory, and memory configured to store data.

Communications device 120 includes a device configured to send and receive information over a communications network. Communications device 120 is configured to provide communicative connections between portable computerized device 110 and a remote server device, for example, over a cellular telecommunications network.

User interface device 130 includes a device or devices configured to provide information to and receive information from a user using the portable computerized device. User interface device can include a touch screen display, a computer monitor, a keyboard device and a mouse device, or any other similar input and output devices known in the art.

Processor device 140 includes a computerized processor and operates computerized processes embodied as one or more computerized modules 142, 144, and 146 including code configured to perform certain functions enabling the disclosed process.

User interface module 142 is configured to provide a display output and interaction screen to a user, process inputs received from a user, and interact with requests for information from a remote server device such as server device 10. User interface module 142 can further interact with other software, for example, retrieving contact information from a contacts list for the smart phone. Choices and wishes module 144 is configured to operate programming related to providing questionnaires to users, accessing user help databases providing more information about different choices that can be made, and managing periodic prompts to the user to update or confirm information. Account management module 146 is configured to manage the account associated with the user, manage billing and payment issues, and store preferences of the user. Module 142, 144, and 146 are exemplary, and a number of different modules dividing tasks to be performed for the disclosed process are envisioned.

Modules in server 10 of FIG. 1 and portable computerized device 110 are exemplary. Tasks performed by these modules can be divided, modified, augmented, or otherwise amended in a number of ways, and the examples provided by the disclosure are not intended to be limiting.

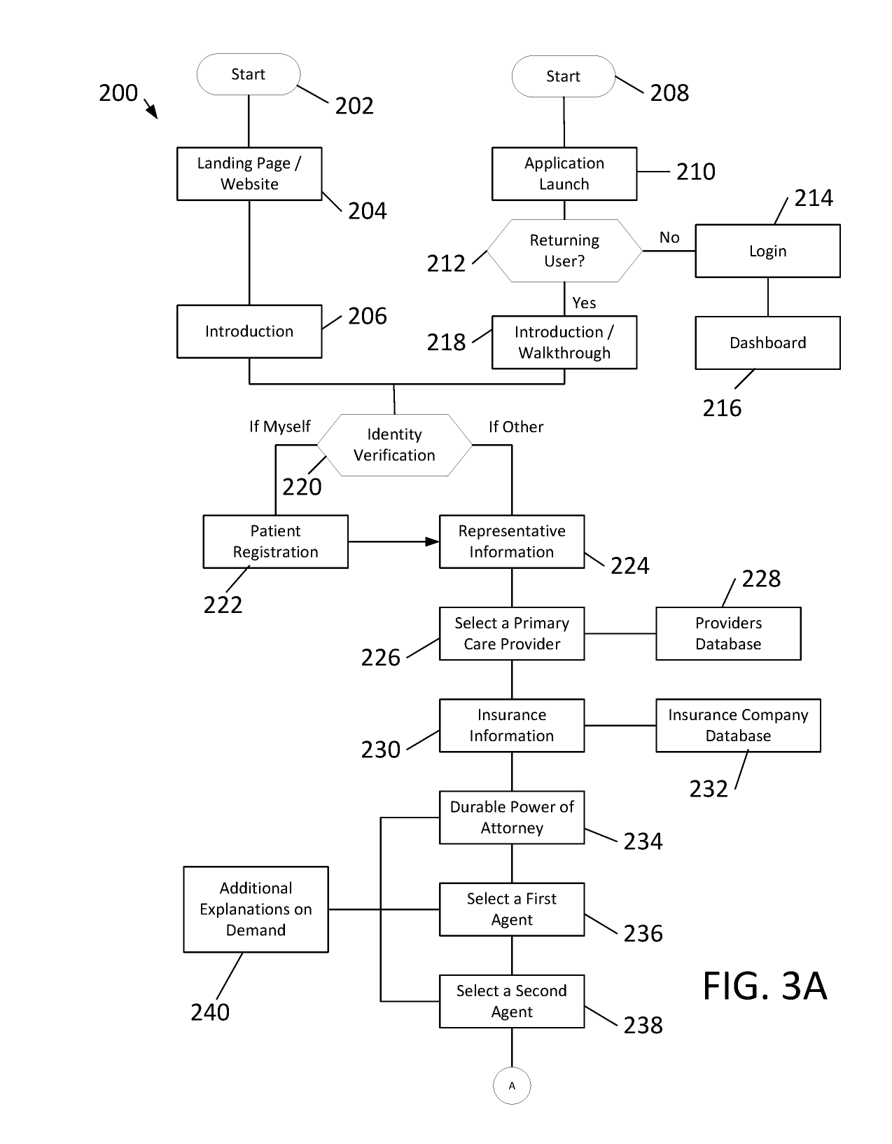

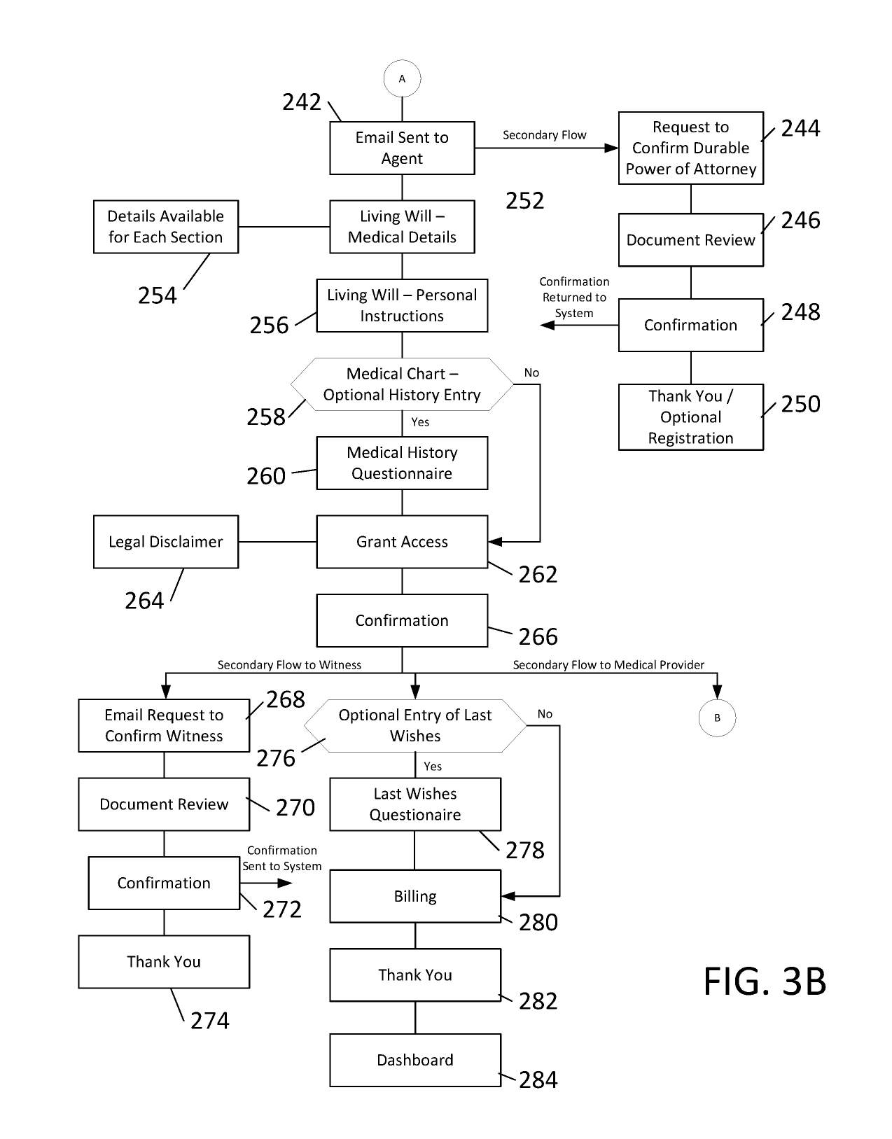

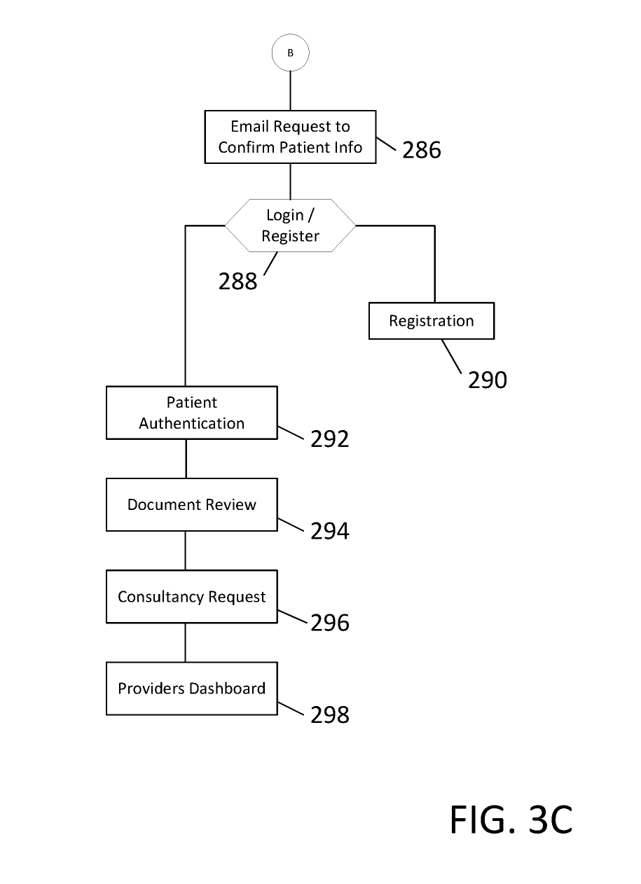

FIGS. 3A-C illustrate an exemplary computerized process configured to operate an advanced care planning process. Process 200 can start in one of two exemplary ways, with step 202 starting from a website or online landing page progressing to step 204 or with step 208 from a smart phone application launch progressing to step 210. For a user utilizing a webpage or online landing page at step 204, the user is provided with an introduction at step 206 and the process advances to step 220. For a user utilizing an application on a smart phone at step 210, at step 212 the process requests information from user whether the user is a returning user. If the user is not a returning user, the process advances to step 218 where the user is provided an introduction or walkthrough of the process, and then the process advances to step 220. If the user is a returning user, then the process advances to step 214, where the user can login, and the process advances to step 216 where the user is provided with a dashboard with a list of options for the user to perform tasks such as auditing and changing choices and wishes already entered during a previous use of the process.

Step 220 prompts the user for identity information to determine whether the user is logging in for his or herself as a customer user or if the user is an agent or care provider logging in for another person. If the user is logging in for him or herself, the process advances to step 222, where the process asks the user questions related to the identity of the user, such as name, date of birth, social security number, address, contact information, marital status, etc. and then progresses to step 224. Step 224 asks information about representatives of the customer user, for example, information related to a designated agent or medical provider for the customer user. Step 226 enables entry of a primary care provider such as a physician. Step 228 enables access of a medical provider database, for example, enabling one to identify a licensed physician or a known clinic or hospital. Step 230 enables entry of insurer information. Step 332 enables access of an insurance company database. Step 234 enables designation of a durable power of attorney, an agent capable of making decisions upon the incapacitation of the customer user. Step 236 enables designation of a first agent enabled to act on behalf of the customer user. Step 238 enables designation of a second agent enable to act on behalf of the customer user, either in parallel to the first agent or in the event the first agent is incapacitated or cannot be contacted. If the user has any questions during the selections of steps 234, 236 and 238, step 240 is provided giving the user access to additional explanations.

At step 242, an email or other electronic communication is sent to the designated agent given durable power of attorney. A request is made at step 244 to the agent to confirm acceptance of durable power of attorney. At step 246, the agent is given an opportunity to review the related documents created by the process. At step 248, the agent confirms/signs documentation confirming the durable power of attorney and it is returned to the process. At step 250, the agent is provided a thank you and is given an optional opportunity to register in the system for ease of later contact.

After step 242, the process proceeds to step 252 where the user is provide choices and wishes related to a living will document. At step 254, details and explanations are provided for any section of the living will document. At step 256, the user is given an opportunity to enter personal instructions related to the living will. At step 258, the user is given an optional ability to enter medical history into a medical history questionnaire. If the user chooses to enter the medical history, the process advances to step 260 where a medical history questionnaire is provided. At step 262, the user is requested to grant access for the information entered in previous steps to agents and medical personnel. Access to a legal disclaimer is provided, explaining the privacy rights of the user and limiting the responsibility of the process provider from responsibility for actions taken by agents given access to the entered information. At step 266, the user is asked to confirm readiness to publish the information provided in the process. Upon confirmation of the user, sub-processes can be initiated. Legal requirements in some states can require a witness for choices and wishes designated in the process. Steps 268, 270, 272, and 274 enable a witness to be contacted, given an opportunity to review the necessary documents, confirm their role as a witness with the required signatures, and provide a thank you to the witness, respectively. Steps 286, 288, 290, 292, 294, 296, and 298 enable a medical provider to be contacted, given an opportunity to login or request registration with the process provider, register with the process provider, authenticate the identity of the user as a patient, review necessary documents provided by the process provider, give the medical provider an opportunity to request consultancy with necessary persons such as the durable power of attorney or the agents, and access a medical provider's dashboard to make other selections relevant to medical providers, respectively.

In step 276, the user is provided with an option to enter other last wishes. If the user selected to enter other last wishes, a last wishes questionnaire is provided at step 278. At step 280, billing/invoice issues related to the user utilizing the process are addressed. The user can be given an opportunity to enter payment information or to request billing by mail. At step 282, a thank you is provided to the user. At step 284, the process provides a user dashboard the same or similar to the dashboard provided in step 216.

The process steps of process 200 are exemplary, and the disclosure is not intended to be limited to the exemplary steps provided.

It will be appreciated that living wills can address a number of medical decisions relative to an incapacitated patient that cannot communicate wishes at that time. For example, living wills can include choices and wishes regarding do not resuscitate wishes, orders regarding use of antibiotics, use of mechanical respiration and CPR devices, kidney dialysis, and other similar procedures.

Last wishes can include choices related to a funeral, cremation or burial, designation of an honorarium or charity, and other similar choices.

Other designations can be made within the process. For example, a will enabling disposition of personal possessions and funds can be entered in the process. Wishes regarding custody of minor children and pets can be communicated. Links including access information to financial funds, life insurance policies, security boxes held at banks, financial trust details, and other similar information can be securely held. An executor can be named.

The process can include a tiered release of information. For example, upon initiation of the process, a user can have an introductory email sent to family members, providing the family members a place to look of important Internet links and information when the user later becomes ill or incapacitated. As the user becomes terminal, a doctor or medical facility designated in the process can be given the opportunity to prompt a second release of information such as last wishes and living will details. As the user passes away, a court can provide authority for release of information that the user designated not to be released until after death.

Details of choices and wishes of a customer user can be sensitive information. For example, a spouse or a child can be upset to learn that the user does not wish to be resuscitated. The process can include a data wall or data vault, enabling release of certain details, such as designation of a preferred physician or hospital that family members can see as needed, but retaining access to closely held information until certain events can be certified to have occurred.

The end of life wishes of the customer user can be held secure according to a number of different methods. The details of the end of life wishes can be released based upon an occurrence of a designated event. Examples of designated events include but are not limited to authorized release by the customer user, authorized release by a designated agent or attorney, or confirmed incapacitation by an agent or a medical professional.

In another example, the customer user can have an email sent to family members, and the email can include a computerized link to authorize release of the end of life wishes. In one example, a family member can authorize release of the end of life wishes by accessing the link (the linked webpage or information can include a confirmation/declaration that some event has taken place.) In another example, a family member clicking on the link may create an email back to the customer user's account confirming or denying access to the end of life wishes. If the customer user does not answer in 30 days, the access may be granted. In another example, a unanimous or majority entry by the family members sent the email may release the end of life wishes. In another example, a death certificate or a court order may be used to authorize release of the end of life wishes. A number of designated events for release of stored end of life wishes can be utilized, and the disclosure is not intended to be limited to the examples provided herein. Data held by the system operating the disclosed process can be held in an encrypted and firewalled system, as are known in the art.

The disclosed process can be used to provide information to an end of life medical care provider after the customer user is no longer able or competent to provide those wishes to the end of life medical care provider. Similarly, the disclosed process can be used to designate a power of attorney agent after the customer user is no longer able or competent to make such a designation. The system is advantageous because it can be operated by a private third party outside of a hospital system, hospice or nursing care system, etc. that would be providing the end of life care. Such a system operated in house at a medical or elder care facility can be difficult to access and periodically edit. Such a facility has no vested interest in providing such access, and most persons do not want to physically visit an elder care facility before they need to. It can be disconcerting to make such visits. The disclosed system enables a user to use a home computer, cellular device, or other similar computerized device to access a computerized server device operating the disclosed process.

The disclosed system enables one to enter end of life wishes including last wishes. Last wishes can be stored or recorded as a video recording, an audio recording, a written message or any other media for recording a message, with the message to be delivered later to a desired audience.

The disclosed system can securely hold in an encrypted and firewalled database the last wishes of the customer user. These details can be released upon any of a number of designated events occur, such as incapacitation of the user, serious infirmity of the user, designation of the user as having a terminal illness, entry of the user into hospice or a nursing home, and death of the user.

The disclosed process can provide the customer user an ability to edit the end of life wishes at a time after an initial entry of the end of life wishes. This can include a periodic reminder to the customer user to edit the end of life wishes.

The disclosed process can include, after monitoring the designation of the end of life wishes, providing a lock screen widget feature which, when activated, provides the customer user with a digital copy of the designated end of life wishes.

The disclosed process can include automatically gathering signatures. This can include determining a list of third party signatures required to enact the designated end of life wishes, automatically sending out forms to be signed based upon the list of third party signatures, and receiving and storing returned signed forms.

The disclosed process can include providing a printed last wishes placard providing directions for how to obtain the end of life wishes. This can include a sticker or card providing notification that last wishes exist and providing a process for requesting access to the last wishes.

The disclosure has described certain preferred embodiments and modifications of those embodiments. Further modifications and alterations may occur to others upon reading and understanding the specification. Therefore, it is intended that the disclosure not be limited to the particular embodiment(s) disclosed as the best mode contemplated for carrying out this disclosure, but that the disclosure will include all embodiments falling within the scope of the appended claims.

1. A computerized process for advanced care planning of a customer user, the process comprising:
within a computerized processor, operating programming configured to:
monitoring designation by the customer user of end of life wishes of the customer user comprising one of:
monitoring entry of a durable power of attorney agent;
monitoring entry of choices related to a living will for the customer user; and
monitoring entry of choices related to last wishes of the customer user;
monitor entry of at least one designee to act on behalf of the customer user;
securely hold the end of life wishes until a designated event occurs; and
release the end of life wishes to the designee upon occurrence of the designated event.

2. The process of claim 1, wherein monitoring entry of the at least one designee comprises monitoring entry of an end of life medical care provider.

3. The process of claim 2, wherein the computerized processor is maintained by an entity separate from the end of life medical care provider.

4. The process of claim 1, wherein monitoring entry of the at least one designee comprises monitoring entry of a person to provide end of life care.

5. The process of claim 1, wherein monitoring designation by the customer user of life wishes of the customer user comprises monitoring entry of the choices related to the last wishes of the customer user comprising recording entry of a recorded video by the customer user; and
wherein releasing the end of life wishes to the designee comprises requesting the designee to publish the recorded video to an intended audience.

6. The process of claim 1, wherein monitoring designation by the customer user of life wishes of the customer user comprises monitoring entry of the choices related to the last wishes of the customer user comprising recording entry of a recorded audio message by the customer user; and
wherein releasing the end of life wishes to the designee comprises requesting the designee to publish the recorded audio message to an intended audience.

7. The process of claim 1, wherein monitoring designation by the customer user of life wishes of the customer user comprises monitoring entry of the choices related to the last wishes of the customer user comprising recording a written message by the customer user; and
wherein releasing the end of life wishes to the designee comprises requesting the designee to publish the recorded written message to an intended audience.

8. The process of claim 1, wherein securely holding the end of life wishes until the designated event occurs comprises holding the end of life wishes until the customer user is incapacitated.

9. The process of claim 1, wherein securely holding the end of life wishes until the designated event occurs comprises holding the end of life wishes until the customer user passes away.

10. The process of claim 1, further comprising providing the customer user an ability to edit the end of life wishes at a time after an initial entry of the end of life wishes.

11. The process of claim 10, wherein providing the customer user the ability to edit the end of life wishes comprises providing a periodic reminder to the customer user to edit the end of life wishes.

12. The process of claim 1, further comprising, after monitoring the designation of the end of life wishes, providing a lock screen widget feature which, when activated, provides the customer user with a digital copy of the designated end of life wishes.

13. The process of claim 1, further comprising:
determining a list of third party signatures required to enact the designated end of life wishes;
automatically sending out forms to be signed based upon the list of third party signatures; and
receiving and storing returned signed forms.

14. The process of claim 1, further comprising providing a printed last wishes placard providing directions for how to obtain the end of life wishes.

15. The process of claim 1, wherein monitoring designation by the customer user of life wishes of the customer user comprises monitoring entry of the choices related to the living will comprising monitoring answers to a series of questions related to one of authorizing or denying future medical treatment for the customer user under varying particular circumstances.

16. The process of claim 15, further comprising providing access to explanations on demand to each of the series of questions.

17. A computerized process for advanced care planning of a customer user, the process comprising:
within a computerized processor, operating programming configured to:
monitoring designation by the customer user of end of life wishes of the customer user comprising:
monitoring entry of a durable power of attorney agent;
monitoring entry of choices related to a living will for the customer user; and
monitoring entry of choices related to last wishes of the customer user;
monitor entry of at least one designee to act on behalf of the customer user;
securely hold the end of life wishes until a designated event occurs; and
release the end of life wishes to the designee upon occurrence of the designated event.

18. The process of claim 17, further comprising providing access to explanations on demand to each of the end of life wishes.

19. A computerized process for advanced care planning of a customer user, the process comprising:
within a computerized processor, operating programming configured to:
monitoring designation by the customer user of end of life wishes of the customer user comprising:
monitoring entry of a durable power of attorney agent;
monitoring entry of choices related to a living will for the customer user; and
monitoring entry of choices related to last wishes of the customer user;
determining a list of third party signatures required to enact the designated end of life wishes;
automatically sending out forms to be signed based upon the list of third party signatures;
receiving and storing returned signed forms;
monitor entry of at least one designee to act on behalf of the customer user;
securely hold the end of life wishes until a designated event occurs; and
release the end of life wishes to the designee upon occurrence of the designated event.
